(19) United States
(12) Patent Application Publication
(10) Pub. No.: US 2010/0022997 A1
(43) Pub. Date: Jan. 28, 2010

(54) Method and process for producing youthful-appearing, small-pored, and smooth skin

(76) Inventor: Diana J. Parsons, Berkeley, CA (US)

Correspondence Address: BUSH INTELLECTUAL PROPERTY LAW;c/o CPA Global, P.O. Box 52050, Minneapolis, MN, 55402, US

(21) Appl. No.: 10/735,362

(22) Filed: Dec. 12, 2003

Related U.S. Application Data
(63) Continuation-in-part of application No. 10/047,335, filed on Jan. 14, 2002, now abandoned.

(51) Int. Cl. A61B 18/20; A61K 31/203; A61P 17/00

(52) U.S. Cl. 606/9; 514/613

(57) ABSTRACT

The method and process for producing youthful appearing, small-pored, and smooth skin comprises pre-treating the skin topically with retinoic acid, applying a contaminant in oil to the skin, and removing the contaminate by exploding or rupturing it with laser light. When treating the face, removing the contaminant requires only about 4 minutes. The laser light itself does not injure the skin but the rupture of the contaminant produces thermal injury in the high dermis leaving the epidermis intact. Repeated intermittent laser treatments and applications of topical retinoic acid produces the desired results in the skin indefinitely as long as the treatment and applications are continued.

This is a Continuation-In-Part Application of application Ser. No. 10/047,355, which is hereby abandoned.

### FIELD OF THE INVENTION

The present invention relates generally to laser treatment of the skin and, more particularly, to a laser treatment inducing a chronic wound in the high dermis, leaving the epidermis intact, with topical pretreatment and post-treatment of the skin with retinoic acid.

### TECHNICAL BACKGROUND

Laser light treatment of the skin is used to rejuvenate the skin, remove pigments and hair, and treat infection. In the field of dermatology and plastic surgery the use of lasers is principally based upon two types of mechanisms: a thermal effect where the laser light energy is converted into heat energy, or a mechanical effect where the laser light energy is converted into shockwaves in the skin.

Most laser treatments use thermal-based laser therapy to vaporize the superficial wrinkles and top layers of the skin so that new collagen and skin can be naturally provided in a healing response. The procedure is performed under either local or general anesthetic, takes a few hours to complete, and recovery takes one to two weeks. During the first week patients may suffer a sensation of intense heat on the skin. Severe burns can occur and result in permanent scarring. Bacterial and yeast infection have been reported and can also lead to scarring. Additional potential complications include changes in pigmentation and herpes infection.

Retinoic acid (Retin-A, tretinoin) has also been used as a topical dermal treatment to improve the appearance and texture of the skin. Retinoic acid thins the stratum corneum, increases the thickness of the epidermis, and increases the production of collagen in the dermis. However, retinoic acid causes skin redness and sensitivity to the sun. Repeated use can cause loss of pigment, painful irritation, dryness, swelling of the skin, and contact dermatitis.

In an effort to overcome these drawbacks of laser therapy and to avoid the unwanted side effects of retinoic acid, Tankovich et al. (U.S. Pat. No. 6,036,684) developed a laser method of photomechanical activation in the skin using a Q switched Nd:YAG laser with a wavelength of 1064 nm and an exposure of 2.5 J/cm². Exposure of the skin to this type of laser treatment by itself has no effect on the skin because the skin has no inherent target at the 1064 nm wavelength. However, when an activating solution of graphite or carbon particles suspended in baby oil is applied to the skin, these particles become the target of this 1064 nm wavelength, exploding when exposed to the laser light. Prior to application of the laser, the particles are forced into the skin, below the surface of the stratum corneum using ultrasound. Thereafter, the explosion of the carbon particles by the laser light produces a localized mechanical injury in the hair follicles and pores of the skin. There is no significant injury to the skin tissue because the laser energy which is not absorbed in the carbon is harmlessly dissipated in the skin. The low fluence of 2.5 J/cm² leaves the epidermis intact and the typical adverse effects of laser treatment do not occur. Although the photomechanical laser process of Tankovich is considerably safer than the standard photothermal laser treatments and leaves the epidermis intact, this photomechanical laser process is relatively ineffective in treating the skin and has not been commercially successful. What is needed is a means for producing adequate new collagen deposition in the high dermis to produce youthful-appearing, small-pored, smooth skin.

### SUMMARY OF THE INVENTION

The present invention provides a process and method for producing skin rejuvenation and therapy by pretreating the skin with retinoic acid, producing a chronic thermal injury wound in the high dermis using photomechanical laser therapy, maintaining skin rejuvenation and therapy chronically by repeating the thermal injury in the high dermis at least once per year, and by topically applying retinoic acid bi-weekly. A Q-switched Nd:YAG laser with a wave length of 1064 nm is used at 2.5 J/cm² to vaporize a topical activating solution of carbon particles suspended in baby oil. A single treatment of the face can be completed in 4 minutes without anesthesia, with no need for a period of recovery, and at a relatively low cost.

An advantage of the present invention is the production of a chronic wound in the high dermis with no damage to the epidermis.

Another advantage of the present invention is the enhancement of laser-induced collagen deposition in the high dermis with retinoic acid.

Another advantage of the present invention is the chronic life-time rejuvenation and therapy of the skin.

Another advantage of the present invention is the removal and prevention of acne by suppression of sebaceous glands and reduction of skin pore size.

Another advantage of the present invention is laser therapy at a low energy level.

Another advantage of the present invention is the use of retinoic acid without side effects.

Another advantage of the present invention is therapeutic removal of infections of the skin.

Another advantage of the present invention is the chronic rejuvenation and therapy of the skin at a relatively low cost.

Another advantage of the present invention is that a single treatment of the skin of the face can be completed within four minutes.

### BRIEF DESCRIPTION OF THE DRAWINGS

FIG. 1 describes the method of the present invention.

FIG. 2 illustrates a cross sectional view of the skin and the role of heat and oil in the method of the present invention.

FIG. 3 describes the process of the present invention.

FIG. 4 shows the effects of the laser method of the present invention on the restoration of the skin of a patient suffering from acne vulgaris.

### DETAILED DESCRIPTION OF THE PREFERRED EMBODIMENTS

While the following description details the preferred embodiments of the present invention, it is to be understood that the invention is not limited in its application to the details of construction and arrangement of the parts illustrated in the accompanying drawings, since the invention is capable of other embodiments and of being practiced in various ways.

Tankovich et al performed skin biopsy studies following their laser/carbon particle treatments. These studies confirmed that there was no significant injury to the skin. However, they observed new collagen fiber formulation in the upper part of the dermis immediately below the epidermal basal membrane. See column 4, lines 1-4 of Tankovich et al. Tankovich et al. had no explanation for this effect. I have studied this effect and have discovered that it is produced by a selective wounding of the high dermis, and that the shorter the wavelength of the laser, the deeper the injury in the dermis. In the Tankovich patent, the extent of the injury in the high dermis resulting from the exploding carbon particles in the epidermis was insufficient to induce adequate collagen deposition during wound healing.

Wound healing in the skin occurs over a year's time after an initial wounding, regardless of the means by which the wounding is induced. In the first five days the wound is comprised of inflammatory cells and new blood vessels. Then an immature form of collagen is laid down parallel to the skin surface. For six months the body shifts this collagen around, trying to identify the strongest repair. Around six months after the initial wounding, this immature collagen is replaced with mature collagen protein oriented perpendicular to the skin surface. Three months into this process, or months ten, eleven, and twelve from the initial wounding, these protein strands cross-link. This effect is visible on the skin's surface as an apparent shrinkage of the size of the scar to one third its original size.

Twelve months from the initial injury, the wound becomes quiescent. However, any additional insult to the wound site during the first six months of repair causes a signal for a stronger permanent repair. Any additional insult to the wound site during the second six months results in additional mature collagen in the wound. As long as some injury is repeated before twelve months has elapsed, the site becomes a chronic wound producing more collagen, undergoing more cross-linking, and more total surface area shrinkage.

The method and process of the present invention described herein outlines a series of treatments using the Tankovich laser and activating solution to maximize the initial wounding signal selectively in the high dermis during the first six months of application. The Tankovich processes disclosed in U.S. Pat. No. 5,423,803 and U.S. Pat. No. 6,036,684 are incorporated herein by reference. However, in the present invention the activating solution does not need to be forced into the spaces of the skin with ultrasound as required by the Tankovich process. In order to produce adequate collagen depositing, beginning at the sixth month from the initial wounding, a series of booster laser treatments is given until the desired skin resurfacing end point is reached. At a minimum, one booster laser treatment is given before twelve months has elapsed to keep the wound active. The overall process requires a plurality of wounding events and thus necessitates at least two laser treatments so that the high dermis may be selectively maintained in a chronically wounded condition, whereby the wound is prevented from healing. If the wound is not kept active, it will be necessary to repeat the initial wounding series of the first six months in order to continue the resurfacing process chronically.

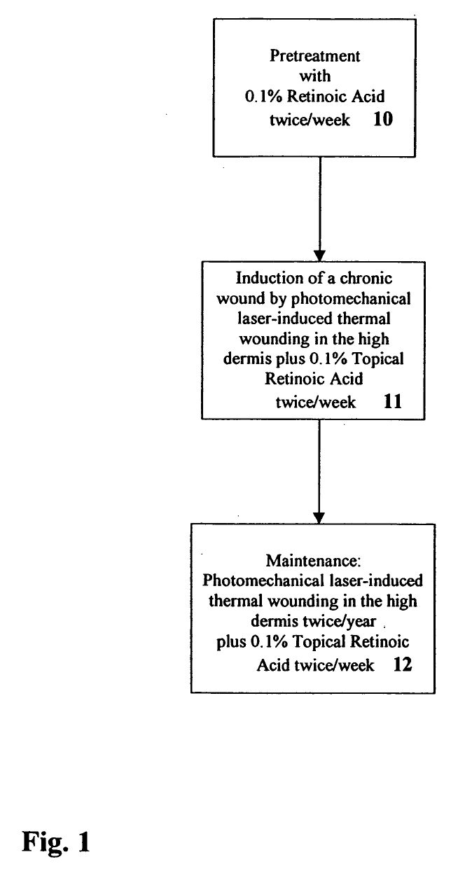

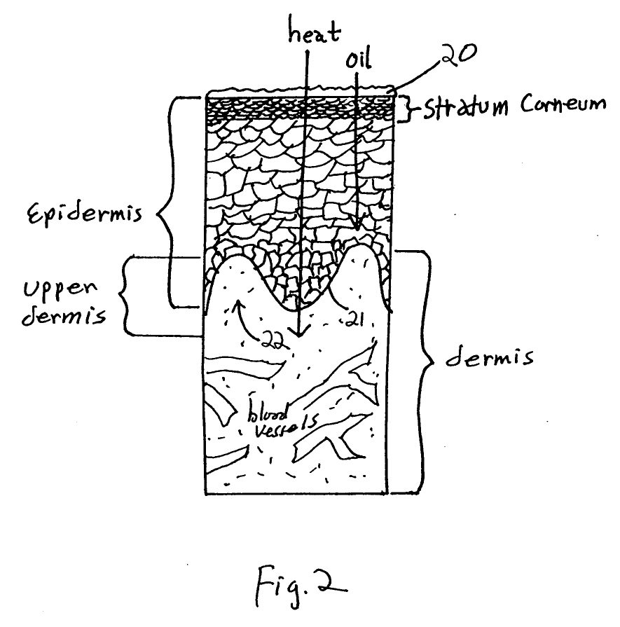

The first step of the method of the present invention is shown in FIG. 1, Step 10, which is a topical pretreatment of the skin with a collagen inducing agent and an angiogenesis inducing agent, preferably, retinoic acid, and in particular its nongeneric form, such as Retin-A (tretinoin). Retinoic acid returns the architecture of the skin back to its more youthful form. As skin ages, the hill and valley nature of the dermalepidermal junction 21 flattens (see FIG. 2). Topical retinoic acid restores the up and down pattern at the junction 21. In youth, the basal cells which originate at the dermalepidermal junction 21 create the layers of the epidermis every three weeks. With aging, this slows to four to six weeks. Topical retinoic acid on the skin returns the process to every three weeks. The topmost layer of epidermal cells, the stratum corneum, is comprised of dead cells held together by a cell glue. With age, this layer thickens making the skin less fresh-appearing and making the pores larger. Topical retinoic acid directly attacks the cell glue, thinning the stratum corneum and making the pores smaller by reducing the amount of debris therein. In my studies I have discovered that 0.05% to 1% concentration of retinoic acid in a topical formulation has the property of inducing new collagen and new blood vessel formation in the high dermis. The pre-treatment period is one to four weeks, preferably two weeks with retinoic acid being applied twice a week with at least two days between each application. The laser therapy of the present invention will not be effective without the pre-treatment application of topical retinoic acid.

The second step 11 in the method of the present invention shown in FIG. 1 is the creation of a chronic wound selectively in the high dermis. This is produced by a photomechanical laser treatment wherein the laser light does not interact directly with the skin but instead interacts with a contaminant in the skin. The contaminant has the properties of absorbing the laser light and exploding. The contaminant is carbon or graphite particles in oil 20 which is applied to the skin (see FIG. 2). Once the contaminant or activating solution is applied to the skin, the laser treatment can begin. The energy from the laser is adjusted to be just sufficient to cause the particles to explode. As the particles explode, they cause the removal of the stratum corneum and the mineral oil 20 penetrates into the epidermis producing hydration of the epidermis by retarding the evaporation of water (see FIG. 2). The heat from the explosion of the contaminant particles will induce a photothermal injury relatively selectively in the rete peg area of the high dermis 22 initiating a normal wound healing process. The epidermis is left intact. In order to produce a sufficient degree of injury to the wound, the laser treatment is produced several times over a six month period, preferably six times over a six month period. During this first six months the retinoic acid is applied topically twice per week as described above.

Due to the use of retinoic acid, the wounding events produce much more collagen than would be produced by such events without the use of retinoic acid. Furthermore, the retinoic acid causes the creation of a blood vessel supply to support the collagen. The build up of new collagen in the high dermis 22 in response to the laser treatment and retinoic acid treatment thickens the skin and increases its turgor resulting in smaller skin pores. Broken blood vessels, angiomata, and ice pick scars or suture marks are also minimized, being crowded out by the new collagen. Easy bruisability of the skin is lessened by the collagen build up. Lips become redder, fine lines are minimized, and dark circles around the eyes are minimized. As the new collagen cross-links major wrinkle lines, such as nasal labial folds and perioral and perioccular lines (caused by the underlying attachment of muscles to the deep dermis), marionette lines, sunken-in corners of the mouth, and forehead, temporal, and glabellar lines, appear to melt away into the new skin. The new skin has a glowing quality resulting from the pulsating nature of the laser injury and the high content of mineral oil in the activating solution. Retinoic acid and laser treatment work together to minimize bound differential pigmentation caused by sun damage and hormones (mylasma). Redness from inflammation, as in acne vulgaris, acne rosacea, or maturing scars or striae is also reduced.

Because of the closeness of the sebaceous glands to the skin surface and because of the new collagen formation which reduces the size of the skin pores, the method of process of the present invention is useful in the treatment of sebaceous gland disorder such as acne. The secretion from the sebaceous glands is reduced and the smaller skin pore size prevents bacteria from entering the skin pores. In addition, heat generated from the explosion of the contaminant particles is sufficient to kill bacteria, fungi, and viruses on the skin's surface or in the high dermis. This also allows treatment of conditions such as, for example, plantar warts, herpes cold sores, athletes foot, psoriasis, and eczema.

The third step 12 in the method of the present invention, depicted in FIG. 1, is the long term continued chronic maintenance of the high dermis in a wounded condition in order to produce a sustained rejuvenation of the skin and treatment of skin conditions. This is accomplished by application of the laser treatment of the present invention at least once a year and preferably twice, along with the continued application of topical retinoic acid to skin twice per week as described above. The maintaining of the high dermis in a chronically wounded condition causes the high dermis to be in a chronic state of repair, whereby new collagen is produced in the skin above the level of inherent collagen. The repeated creation of new collagen is beneficial since the inherent collagen has usually been damaged by sunlight.

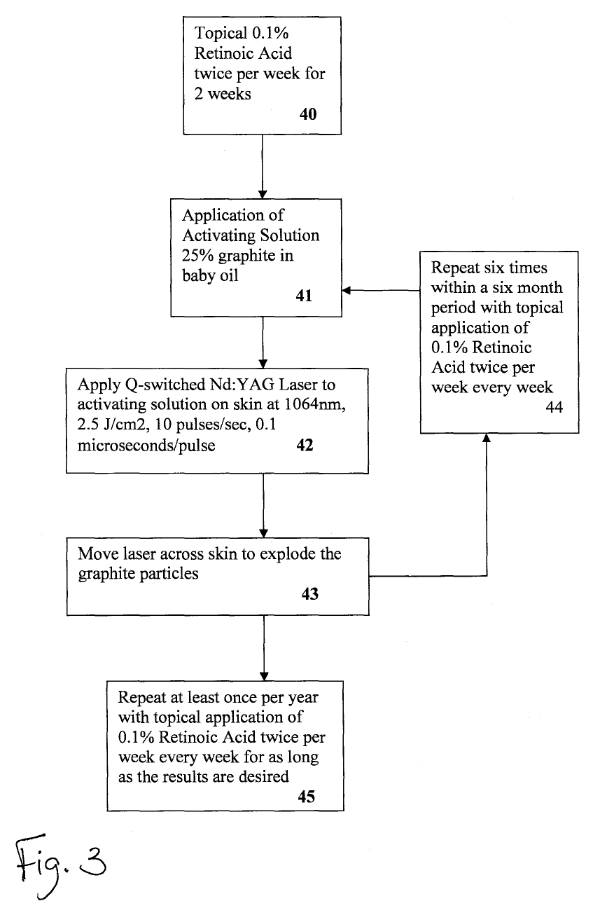

The process of the present invention for treating the skin is shown in FIG. 3. In the first step 40, pre-treatment is initiated with topical retinoic acid applied to the skin at least twice per week. The concentration of retinoic acid in the topical formulation is between about 0.05% to 1% preferably about 0.1%. The pre-treatment period is, preferably, two weeks. In the next step 41, an activating solution of graphite in baby oil is applied to the skin. The graphite-oil ratio may range from about 1:1 to 1:9, preferably 1:4, i.e., about 20 percent graphite suspended in about 80 percent oil by weight. In the next steps 42 and 43 a laser beam is scanned over the area treated with the activating solution so as to clean substantially all of the mixture from the skin surface by exploding or fracturing the carbon or graphite particles in the oil. This scanning process takes from about 2 to 10 minutes to complete on the face, usually about 4 minutes.

It is preferred to use a Q-switched neodymium:yttrium-aluminum-gamet (Nd:YAG) laser for the process of the present invention. The wave length of the radiation may range from about 800 nM to 1200 nM, preferably about 1064 nM. The frequency of the pulses from the laser range from about 1 to 20 per second, preferably, about 10 per second. The duration of each pulse ranges from about 0.001 to 1 microsecond, preferably about 0.1 microsecond. The fluence or exposure of the skin treated with activating solution ranges from about 1 to 3 J/cm².

In the next step 44 the laser process is repeated several times to create a chronic wound in the high dermis 22 (see FIG. 2). The process may be repeated 2 to 12 times within a six month period, preferably six times. During this time retinoic acid is applied to the skin 1 to 4 times per week, preferably 2 times per week, with 2 to 3 days between applications. The application of retinoic acid may be varied in an equivalent manner on a monthly basis, i.e., 4 to 16 applications per month, preferably 8 applications per month, where the applications may all be applied within one or two weeks of a given month, but preferably, applied each week of a month.

In the next step 45 the laser process is repeated at least once in the next six months and thereafter at least once per year, but, preferably twice per year. The yearly treatments will maintain a chronic wound in the high dermis. At the same time, topical retinoic acid is applied weekly to the skin as described above. The continued combination of intermittent laser therapy and topical retinoic acid will maintain the rejuvenation and therapeutic results in the skin indefinitely.

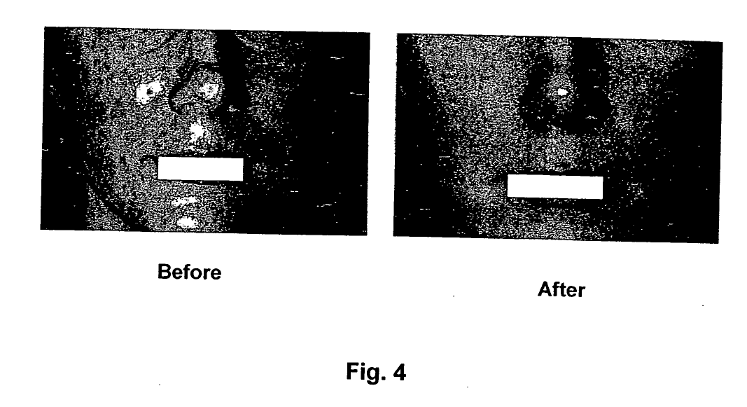

FIG. 4 shows, by way of example, the effects of the laser method of the present invention on the skin of a patient suffering from acne vulgaris. The before picture presents the patient's skin prior to treatment and the after picture shows the patient's skin after the first six months of treatment of the skin with the method and process of the present invention. The lesions and scars are substantially reduced in severity and the process of acne formation has been completely prevented. Furthermore, this has been accomplished leaving the epidermis intact and normal. The process enables the epidermis to be left intact through the use of the Q-switched laser described above. Because of the laser's wavelength, the pulses of heat emitted by the laser are deposited in the high dermis. The low energy of the laser causes those pulses not to burn the epidermis. Continued chronic treatment with the method of the present invention is expected to provide further improvement in the appearance of the skin.

The foregoing description has been limited to specific embodiments of this invention. It will be apparent, however, that variations and modifications may be made by those skilled in the art to the disclosed embodiments of the invention, with the attainment of some or all of its advantages and without departing from the spirit and scope of the present invention. For example, agents other than retinoic acid which induce collagen and blood vessel growth may be used in place of retinoic acid. Any laser system capable of exploding or rupturing a contaminant in the skin without injuring the skin directly can be used in the present invention. The activating solution can be made with any suitable oil with any suitable contaminant.

It will be understood that various changes in the details, materials, and arrangements of the parts which have been described and illustrated above in order to explain the nature of this invention may be made by those skilled in the art without departing from the principle and scope of the invention as recited in the following claims.

1-20. (canceled)

21. A process for producing sustained skin rejuvenation, comprising
a) initially applying retinoic acid to the skin;
b) exploding a contaminant on the surface of the skin with laser light, said laser light having a pulse duration of about 0.001 to about 1 microsecond, a wave length of from about 800 nm to 1200 nm, and a fluence of from about 1 to 3 J/cm², and wherein said exploding a contaminant on the surface of the skin leaves the epidermis intact and normal and produces a wound in the high dermis,
c) applying retinoic acid to the skin 4 to 16 times per month after step b); and
d) repeating step b) at least once every 6 months during step c), wherein steps c) and d) produce a sustained rejuvenation of the skin.

22. (canceled)

23. The process of claim 21 wherein the step of initially applying retinoic acid to the skin comprises two weeks.

24. The process of claim 21 wherein exploding a contaminant on the surface of the skin of the face with laser light is completed within about two to ten minutes.

25. The process of claim 24 wherein exploding a contaminant on the surface of the skin of the face with laser light is completed within about four minutes.

26. The process of claim 21 wherein the step of exploding a contaminant on the surface of the skin with laser light after initially applying retinoic acid to the skin is performed two to twelve times within a six month period.

27. The process of claim 26 wherein the step of exploding a contaminant on the surface of the skin with laser light after initially applying retinoic acid to the skin is performed six times within a six month period.

28. The process of claim 21 wherein said laser light has a pulse frequency of from about 1 to 20 per second.

29. The process of claim 21 wherein said laser light has a wave length of about 1064 nM, a pulse frequency of about 10 per second, a pulse duration of about 0.01 microsecond, and a fluence of about 2.5 J/cm².

30-32. (canceled)

33. The process of claim 21 wherein said retinoic acid is applied topically at a concentration of from about 0.05% to 1% without producing side effects.

34. The process of claim 33 wherein said retinoic acid is applied topically at a concentration of about 0.1%.

35. The process of claim 21 wherein said process is used to improve the appearance of broken blood vessels, angiomata, scars, lines, wrinkles, pigmentation, and redness in the skin.

36. The process of claim 21 wherein said process is used on acne, warts, cold sores, eczema, psoriasis, and infections of the skin.

37. The process of claim 29 wherein said laser light is produced by a Q switched Nd:YAG laser.

38. The process of claim 21 wherein exploding said contaminant on the surface of the skin produces a wound in the rete peg area of the high dermis.

39. (canceled)

40. (canceled)
